(19) United States
(12) Patent Application Publication
(10) Pub. No.: US 2011/0077278 A1
(43) Pub. Date: Mar. 31, 2011

(54) Antifungal compositions and methods

(75) Inventors: Roger Smith, Branchburg, NJ (US); Kamlesh Gaglani, Belle Mead, NJ (US)

(73) Assignee: Troy Corporation

(21) Appl. No.: 12/586,981

(22) Filed: Sep. 30, 2009

(51) Int. Cl. A01N 43/80; A61P 31/10

(52) U.S. Cl. 514/373

(57) ABSTRACT

A combination product including a mixture of 3-iodpropynyl-N-n-butylcarbamate (“IPBC”) and 2-n-N-butyl-1,2-benzisothiazolin-3-one (“BBIT) for use as a fungicide and growth inhibitor in industrial applications. Combination products of the present invention are effective for inhibiting the growth of microorganisms, and exhibit a synergistic antifungal effect against fungi such as, for example, Aspergillus niger, Penicillium funiculosum, Trichoderma virens, Chaetosphaeridium globosum, Penicillium sp., and Caratocystis Pilifera. Methods for using the product in metal working fluids, dry film coatings, in-can preservatives, caulks, sealants leather, wood, rope, cordage, textiles, ink, adhesives and polymeric materials are also described.

### BACKGROUND OF THE INVENTION

1. Field of the Invention

The present invention relates to synergistic compositions of matter and methods for inhibiting the growth of fungus, bacteria, and other microbial agents.

2. Description of the Related Art

Exterior surfaces, interior surfaces and substrates of all types are prone to attack by fungal, algal, bacterial, and protozoan microorganisms when exposed to moisture under mild conditions. Materials which benefit from the protection of a suitable antimicrobial composition for controlling microorganisms include, for example, paints, coatings, stucco, concrete, stone, cementaceous surfaces, wood, caulking, sealants, textiles, rope, leather, paper pulp, ink, adhesives, metal working fluid, fuels, starches, resins, and polymers.

Two or more species of microorganisms are frequently found in proximity with a particular surface or substrate, and the damage caused by various microorganisms acting simultaneously or in series can be especially significant. Consequently, broad spectrum microbicides that can protect against a range of microorganisms are of great commercial interest.

Microbicides which contain a halopropargyl moiety, and especially an iodopropargyl moiety, are disclosed in U.S. Pat. Nos. 3,660,499; 3,923,870; 4,259,350; 4,592,773; 4,616,004 and 4,639,460, to name a few. Included within this class of microbicides is 3-iodpropynyl-N-n-butylcarbamate (hereinafter referred to as “IPBC”). IPBC is renowned as a broad spectrum fungicide. In addition to its fungicidal activity, IPBC has been associated with algicidal activity, as described in Great Britain Patent No. 2,138,292 and U.S. Pat. Nos. 4,915,909 and 5,082,722.

Alone or in combination with other microbicides, IPBC is recognized as a proven fungicide for metalworking fluids; polymers; leather; textiles; wood; and dry film coatings such as paint, stain, and stucco. For example, U.S. Pat. No. 3,923,870 describes the use of IPBC as a fungicide for coating compositions.

Another class of microbicides, designated N-alkyl-1,2-benzisothiazolin-3-ones, are known for fungicidal activity and bactericidal activity. For example, GB Patent No. 1 531 431, issued to Buckley et al., describes the use of N-alkyl-1,2-benzisothiazolin-3-ones for protecting paint films from fungal attack. As another example, U.S. Pat. No. 6,005,032, issued to Austin, describes 2-n-butyl-1,2-benzisothiazolin-3-one (hereinafter referred to as “BBIT”) as a preferred fungicide for protecting plastic materials.

U.S. Pat. No. 6,861,395, issued to Eastwood et al., describes the use of BBIT for inhibiting the growth of microorganisms in a metal working fluid and notes that BBIT exhibits synergy in the presence of sodium omadine and certain fungi. The '395 patent also presents comparative compositions including metal working fluids and IPBC, and reports that BBIT is considerably more stable to high temperature storage than IPBC. The '395 patent is silent regarding any combination of BBIT and IPBC.

An antimicrobial composition comprising IPBC and 1,2-benzisothiazolin-3-one in a ratio which reportedly exhibits synergism is described in U.S. Pat. No. 5,219,875, issued to Sherba et al. The '875 patent is silent regarding BBIT.

While the above described microbicidal combinations are satisfactory for many applications, the preservative industry would welcome an improved microbicidal combination which is relatively more affordable and effective for protecting against a variety of fungi and other microorganisms. Ideally, the improved combination conserves resources and protects the environment by employing relatively small amounts of biologically active materials in an efficient manner.

### SUMMARY OF THE INVENTION

It has now been discovered that IPBC and BBIT act synergistically to inhibit fungi, and can be combined to produce a reliable and broadly effective antifungal composition for industrial applications. Compositions of the present invention include a combination of IPBC and BBIT. The inventive combinations provide better antifungal protection than either of IPBC or BBIT acting alone. Compositions and methods of the present invention can provide the same or better biocidal effect, while utilizing relatively less active material as compared to traditional compositions and methods.

In one aspect, the invention is a composition of matter which comprises IPBC and BBIT in a proportion that exhibits a synergistic antifungal effect. For example, the synergistic antifungal effect has been observed with fungi such as Aspergillus niger, Penicillium funiculosum, Trichoderma virens, Chaetosphaeridium globosum, Penicillium sp., and Caratocystis Pilifera

In another aspect, the invention is a method for protecting a substrate from fungal infection. The method comprises treating the substrate with a synergistic, inhibitory amount of a composition which includes IPBC and BBIT, and exhibits a synergistic antifungal effect.

In still another aspect, the invention is a method for inhibiting the growth of fungi in a metal working fluid. The method comprises adding IPBC and BBIT to the metal working fluid, in order to produce a protected metal working fluid which exhibits a synergistic antifungal effect.

In yet another aspect, the invention is a method for inhibiting the growth of fungi in on or a polymeric material. IPBC and BBIT are incorporated in or on the polymeric material as a fungicide combination which exhibits a synergistic effect.

In still yet another aspect, the invention is a method for protecting a dry film from fungal attack, by adding IPBC or BBIT to a film-forming coating precursor, and exposing the coating precursor to an oxygen-containing gas to form a dry film coating.

### BRIEF DESCRIPTION OF THE DRAWING

FIG. 1 depicts a stacked-axis Cartesian co-ordinate system. Increasing distance on the horizontal axis is proportional to BBIT concentration in weight percent, and inversely proportional to IPBC concentration in weight percent. Increasing distance on the vertical axis is proportional to minimum inhibitory concentration (MIC) against Aspergillus niger, expressed in weight parts per million for the total amount of BBIT plus IPBC. In each composition, the active material is BBIT, IPBC, or a combination of the two. The total amount of active material is 40 weight percent for each of the compositions. The data of FIG. 1 demonstrates that MIC against Aspergillus niger for these compositions is a function of active material composition, and has a minimum value.

### DETAILED DESCRIPTION OF PREFERRED EMBODIMENTS

In a preferred embodiment, the invention is a fungicide comprising IPBC and BBIT. The inventive combination is surprisingly effective for inhibiting the growth of fungi. The invention can be applied to produce a greater antifungal effect or, alternatively, a comparable antifungal effect with relatively less of the active materials.

The inventive combination exhibits a synergistic effect in all observed proportions against the fungus Apergillus niger. Proportions of IPBC to BBIT greater than about 0.3 are preferred; proportions in the range of about 0.6 to about 7 are more preferred, and proportions in the range of about 1 to about 3 are most preferred.

The antifungal synergy of the invention makes it particularly effective against various fungi which are encountered indoors and outdoors. In practice, antifungal compositions are often called upon to protect against one or more unidentified fungus that are encountered in a particular application. To the extent that fungal growth as a whole is inhibited in the particular application, the antifungal composition is considered successful. The identity of the inhibited fungi may remain undetermined.

The invention has been found synergistically effective against a number of precisely identified fungi in the laboratory such as, for example, Aspergillus niger, Penicillium funiculosum, Trichoderma virens, Chaetosphaeridium globosum, Penicillium sp., or Caratocystis Pilifera. It is presumably also effective against as yet unidentified microbes which may proliferate indoors or outdoors. Without intending to limit the scope of the invention in any way, it is expected that the invention will inhibit the growth of many objectionable surface fungi that are capable of causing discoloration.

The inventive combination can be formulated as a ready-to-use mixture in which the weight of IPBC and BBIT totals about 0.5 to about 2.0 wt % of the weight of the mixture. Alternatively, the inventive combination may be in the form of a concentrate which preferably includes more than about 2 wt %, more preferably more than about 10 wt % of IPBC and BBIT. The ready-to use mixture and the concentrate may optionally include additional active materials, as well as components such as surfactants, emulsifiers, wetting agents, pH buffers, and the like. Preferred additional active materials include other isothiazolinones, such as 2-n-octyl-4-isothiazolin-3-one; 4,5-dichloro-2-n-octyl-4-isothiazolin-3-one; 5-chloro-2-methyl-4-isothiazolin-3-one; 2-methyl-4-isothiazolin-3-one; 2-methyl-4,5-trimethylene-4-isothiazolin-3-one; 2-n-octyl-1,2-benzisothiazolin-3-one; 2-n-hexyl-1,2-benzisothiazolin-3-one and 1,2-benzisothiazolin-3-one. Either the ready-to-use mixture or the concentrate may be in the form of a solution, dispersion, or emulsion. The ready-to use mixture and the concentrate may include carriers.

Preferred carriers for the inventive combination include glycol ethers and esters, such as propylene glycol n-butyl ether, propylene glycol tert-butyl ether, 2-(2-methoxymethylethoxy)-tripropylene glycol methyl ether, propylene glycol methyl ether, dipropyleneglycol methyl ether, tripropylene glycol methyl ether, propylene glycol n-butyl ether and the esters of these compounds. Other preferred carriers are n-methyl pyrrolidone, n-pentyl propionate and dibasic esters of several dicarboxylic acids and mixtures thereof. More preferably, the carriers are propylene glycol n-butyl ether, 1-methoxy-2-propanol, and the dibasic isobutyl ester blend of succinic, glutaric and adipic acids. Most preferably, the carriers are those which are low in volatile organic carbon (hereinafter referred to as “VOC”).

When preparing formulations of the present invention for specific applications, the composition also will likely be provided with other conventional components, such as organic binding agents, additional fungicides, auxiliary carriers, processing additives, fixatives, plasticizers, UV-stabilizers or stability enhancers, water soluble or water insoluble dyes, color pigments, siccatives, corrosion inhibitors, antisettling agents, anti-skinning agents and the like. Additional fungicides which may be used in the composition are preferably soluble in the carrier.

According to the present invention, substrates are protected from infection by fungal and bacterial organisms by treating the substrate with a composition of the present invention. Such treating may involve mixing the composition with the substrate, coating or otherwise contacting the substrate with the composition.

In another preferred embodiment, the composition is a metal working fluid which contains a synergistic combination of IPBC and BBIT. For the present purposes, “metal working fluid” includes without limitation water-based fluids, straight oils, quenching fluids, casting fluids and especially soluble oil, semi-synthetic or synthetic metal working fluids. Typically, synthetic metal working fluids comprise an emulsion of one or more synthetic lubricant(s) in an aqueous medium. Suitable synthetic lubricants include glycols such as polyoxyalkylene glycols and glycol esters. The teachings of U.S. Pat. No. 6,861,395 with respect to metal working fluids, and specifically with respect to the use of N-alkyl benzisothiazolin-3-one for inhibiting the growth of microorganisms in a metal working fluid, are hereby incorporated by reference.

In yet another preferred embodiment, the composition is a combination of IPBC and BBIT which optionally includes a monomer or a plasticizer, and is suitable for use in plastic or polymeric materials, such as polyvinyl chloride or polyethylene. The teachings of U.S. Pat. No. 6,005,032, issued to Austin, with respect to protecting plastics from fungal attack, and specifically with respect to the use of N-alkyl benzisothiazolin-3-one as a for plastics, are hereby incorporated by reference.

In other preferred embodiments, the invention composition is suitable as an additive for inclusion in a coating, such as a paint or a stain; or as a preservative for wood, leather, cordage or textiles. The inventive composition may be an in-can preservative for protecting film-forming coating precursors from fungal attack, or a dry film coating preservative to protect a fully-formed coating film from fungus.

The following examples are presented to better communicate the invention, and are not intended to limit the invention in any way. Unless otherwise indicted, all references to parts, percentages or proportions are based on weight.

### Example 1

### Effectiveness of IPBC and BBIT Compositions Against Aspergillus niger

Several antifungal compositions containing IPBC, BBIT or both were investigated, as shown below in Table 1A. Each of the antifungal compositions included a total of 40 wt % fungicidally active material. A commercially available material which contained 95 wt % BBIT was utilized as a starting ingredient and diluted with alcohol as a solvent to prepare the antifungal compositions. Another commercially available material which contained 40 wt % of IPBC, with the balance being mainly aromatics solvents, was also utilized as a starting ingredient and diluted with alcohol as necessary to prepare the antifungal compositions.

Minimum Inhibitory Concentration (hereinafter referred to as “MIC”) for each of the antifungal compositions was determined against a spore suspension of the fungus Aspergillus niger (ATTC 6275) with the aid of an Autoplate 4000 spiral plater commercially available from Spiral Biotech, Inc., Norwood, Mass., and its accompanying spiral gradient endpoint software (hereinafter referred to as “the SGE software”). The Autoplate 4000 automates the normal serial dilution method for determining MICs. The automated method employs a simplex lattice design with the antifungal compositions.

Spore suspensions for the test fungus were prepared by growing Aspergillus niger on a Difco malt agar slant in an incubator for 1 week at 28° C. Spores were loosened by adding a small amount of buffer solution at pH 7.0 and scraping with a sterile nichrome wire loop. This process was repeated twice. The buffer solution included phosphate buffer and magnesium chloride, and was obtained commercially from Thomas Scientific Company, as Lot # 023-0703.

Loosened spores were removed from the slant by aseptically pouring them into a sterile bottle containing 30 ml of the buffer solution and a volume of approximately 40 ml of 6 mm diameter borosilicate glass beads. The bead bottle was shaken to disperse the spores and adjusted to a final liquid volume of 50 ml. For use as a test inoculum, spore density was adjusted in distilled water blanks to that of a 0.5 McFarland nephelometer standard.

The Autoplate 4000 automatically applied 54.3 micro-liters of each antifungal composition of interest to the surface of 150 MM malt agar plates using an exponential application gradient. Antifungal composition concentration was heaviest near the center of the Petri plates and decreased toward the edges. Antifungal composition gradients were allowed to air dry at room temperature for 1 to 4 hours at 23° C. before inoculation with fungi. Spiral gradient plates were inoculated by streaking with cotton swabs that had been soaked in test fungus spore suspension. Streaks were applied in a radial pattern, using a paper template generated by the SGE software to guide the application. Eight radii were inoculated per Petri plate. Each radius is considered as one replicated observation.

Inoculated spiral gradient plates were incubated for 48 hours in an incubator at 28° C. Visible growth of the test fungus developed along the radial streaks and ended where the concentration of the antifungal composition was sufficient to prevent growth. This growth endpoint value (expressed in mm as measured from center point of the Petri plate) was used by to compute MIC for the mixture, expressed as parts per million of active fungicide(s). Results are presented in Table 1A below.

TABLE 1A
Fungicide Mixtures and MIC for Aspergillus niger

| | | | | MIC |
|---|---|---|---|---|
| | Composition | BBIT | IPBC | (ppm, average of |
| Trial Number | Number | (wt %) | (wt %) | 8 replications) |
| 1 INVENTION | 1 | 30 | 10 | 0.397 |
| 2 INVENTION | 2 | 22 | 18 | 0.285 |
| 3 INVENTION | 3 | 20 | 20 | 0.303 |
| 3 (retest) INVENTION | 3 | 20 | 20 | 0.302 |
| 4 INVENTION | 4 | 18 | 22 | 0.251 |
| 5 INVENTION | 5 | 10 | 30 | 0.274 |
| 6 (for Q_{A}) COMPARISON | 6 | 40 | 0 | 0.791 |
| 7 (for Q_{A} retest) | 6 | 40 | 0 | 0.910 |
| COMPARISON | | | | |
| 8 (for Q_{B}) COMPARISON | 7 | 0 | 40 | 0.424 |
| 9 (for Q_{B} retest) | 7 | 0 | 40 | 0.436 |
| COMPARISON | | | | |

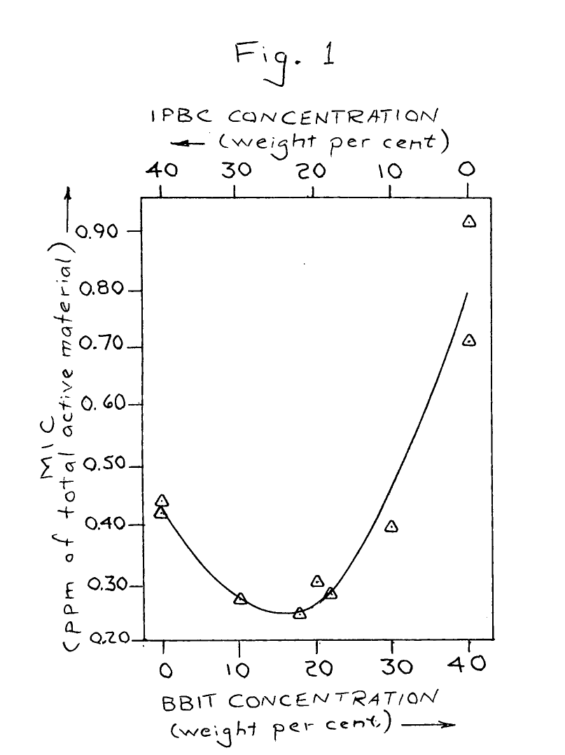

The data of Table 1A is portrayed graphically in FIG. 1, where MIC is depicted as a function of active material composition. FIG. 1 depicts a stacked-axis Cartesian co-ordinate system in which increasing distance on the vertical axis is linearly proportional to minimum inhibitory concentration (MIC) expressed in weight parts per million of active material in a composition, based on the weight of the composition. For a particular composition, the active material in the composition is BBIT, IPBC, or a combination of the two. Increasing distance on the horizontal axis is linearly proportional to the BBIT concentration, expressed in percent BBIT based on the weight of the composition, as indicated on the lower axis of FIG. 1.

For convenience, the percentage of IPBC (which is inversely proportional to increasing distance on the horizontal axis) is indicated on the upper axis of FIG. 1. Because each of the mixtures for which this data is recorded contains a total of 40 wt % BBIT and/or IPBC, distance on the horizontal axis is inversely proportional to IPBC concentration in weight parts per million. In other words, as the horizontal axis is traversed, each additional incremental unit of BBIT is compensated by the absence of a corresponding unit of IPBC.

For the co-ordinate system in FIG. 1, “additive combination” means a fungicide combination for which the trace of MIC is a straight line over a range of BBIT percentages or IPBC percentage. “Synergistic combination” means a fungicide combination for which the MIC trace is concave over a percentage range of either of the active materials. “Antagonistic combination” means a fungicide combination for which the MIC trace is convex of concentration for either of the active materials.

Inspection of FIG. 1 indicates that IPBC and BBIT is a synergistic combination in all proportions, more synergistic for proportions of IPBC to BBIT in the range of at least about 0.3; and still more synergistic for proportions in the range of about 0.6 to about 7. Based on the data portrayed in FIG. 1, the proportion of IPBC to BBIT for maximal antifungal activity against Aspergillus niger is about 1.7.

“Synergistic effect” means the response of a mixture of two or more components which is greater than the sum of the response of the individual components. A mathematical approach for assessing synergy is reported by F. C. Kull, P. C. Elisman, H. D. Sylwestrowicz and P. K. Mayer, in Applied Microbiology, 9:538 (1961). For binary mixtures, the degree of synergistic effect for a range of proportions can be quantified in terms of its “Synergistic Index”, defined by the following equation.

Synergistic Index=Qₐ/Q_{A}+Q_{b}/Q_{B} [Equation No. 1]

where
Qₐ=the quantity of component A used in a binary mixture that gives the desired effect
Q_{A}=the quantity of component A which when used alone gives the desired effect
Q_{b}=the quantity of component B used in a binary mixture that gives the desired effect
Q_{B}=the quantity of component B which when used alone gives the desired effect

Using these criteria, Synergistic Index against Aspergillus niger was calculated for several mixtures of BBIT and IPBC. The desired effect was minimal inhibition of fungus growth. The results of these calculations are presented in Table 1B below.

TABLE 1B

| | Compo- | | | | | | | Syner- | |
|---|---|---|---|---|---|---|---|---|---|
| | sition | | | | | Qₐ/ | Q_{b}/ | gistic | |
| Trial | Number | Qₐ | Q_{A} | Q_{b} | Q_{B} | Q_{A} | Q_{B}/ | Index | Comment |
| 1 INVENTION | 1 | 0.28 | 0.85 | 0.1 | 0.42 | 0.33 | 0.24 | 0.57 | Synergy |
| 2 INVENTION | 2 | 0.16 | 0.85 | 0.13 | 0.42 | 0.19 | 0.31 | 0.50 | Synergy |
| 3 INVENTION | 3 | 0.15 | 0.85 | 0.15 | 0.42 | 0.18 | 0.36 | 0.54 | Synergy |
| 3(retest) INVENTION | 3 | 0.15 | 0.85 | 0.15 | 0.42 | 0.18 | 0.36 | 0.54 | Synergy |
| 4 INVENTION | 4 | 0.11 | 0.85 | 0.14 | 0.42 | 0.13 | 0.33 | 0.46 | Synergy |
| 5 INVENTION | 5 | 0.07 | 0.85 | 0.20 | 0.42 | 0.08 | 0.48 | 0.56 | Synergy |

The Synergistic Indexes shown in Table 1B for compositions of the invention are evidence that mixtures of IPBC and BBIT over a relatively wide range of proportions exhibit a synergistic effect against Aspergillus niger.

### Example 2

### Effectiveness of IPBC and BBIT Compositions Against Various Fungal Species

A synergistic antifungal effect was demonstrated for IPBC and BBIT mixtures against several fungal species (in addition to Aspergillus niger, which is reported above in Example 1). Specifically, the MIC's of three different antifungal compositions (one containing IPBC, one containing BBIT and one containing both) were respectively determined for the fungi Penicillium funiculosum (ATCC 11797); Trichoderma virens (ATCC 9645); Chaetosphaeridium globosum (ATCC 6205); Penicillium sp. (ATCC 12667); and Caratocystis Pilifera (ATCC 15457)

For each of these fungal species, MIC was determined with IPBC as the sole active ingredient, with BBIT as the sole active ingredient, and for a antifungal combination which contained both IPBC and BBIT. Starting ingredients essentially identical to those described above in Example 1, an Autoplate 4000 spiral plater, and software commercially available from Spiral Biotech, Inc., Norwood, Mass., were utilized. Spore suspensions for the fungus were prepared by the procedure described above in Example 1. Results are presented in Table 2A below.

TABLE 2A
Fungicide Mixtures and MIC

| | Compo- | | | | MIC |
|---|---|---|---|---|---|
| | sition | | IPBC | BBIT | (ppm, average of |
| Trial Number | Number | Fungus | (wt %) | (wt %) | 3 replications) |
| 10 INVENTION | 8 | Penicillium | 17 | 10 | 0.25 |
| | | funiculosum | | | |
| 10 (for Q_{A}) | 9 | Penicillium | 27 | 0 | 0.23 |
| COMPARISON | | funiculosum | | | |
| 10 (for Q_{B}) | 10 | Penicillium | 0 | 27 | 0.48 |
| COMPARISON | | funiculosum | | | |
| 11 INVENTION | 8 | Trichoderma | 17 | 10 | 0.6 |
| | | virens | | | |
| 11 (for Q_{A}) | 9 | Trichoderma | 27 | 0 | 0.7 |
| COMPARISON | | virens | | | |
| 11 (for Q_{B}) | 10 | Trichoderma | 0 | 27 | 1.2 |
| COMPARISON | | virens | | | |
| 12 INVENTION | 8 | Chaetosphaeridium | 17 | 10 | 2.2 |
| | | globosum | | | |
| 12 (for Q_{A}) | 9 | Chaetosphaeridium | 27 | 0 | 5.4 |
| COMPARISON | | globosum | | | |
| 12 (for Q_{B}) | 10 | Chaetosphaeridium | 0 | 27 | 1.5 |
| COMPARISON | | globosum | | | |
| 13 INVENTION | 8 | Penicillium sp. | 17 | 10 | 0.2 |
| 13 (for Q_{A}) | 9 | Penicillium sp. | 27 | 0 | 0.29 |
| COMPARISON | | | | | |
| 13 (for Q_{B}) | 10 | Penicillium sp. | 0 | 27 | 0.47 |
| COMPARISON | | | | | |
| 14 INVENTION | 8 | Caratocystis | 17 | 10 | 0.6 |
| | | Pilifera | | | |
| 14 (for Q_{A}) | 9 | Caratocystis | 27 | 0 | 0.42 |
| COMPARISON | | Pilifera | | | |
| 14 (for Q_{B}) | 10 | Caratocystis | 0 | 27 | 2.4 |
| COMPARISON | | Pilifera | | | |

The MIC data presented in Table 2A is employed with Equation No. 1, as described above in Example 1, to calculate the Synergistic Index for each Trial of three compositions. The results are shown below in Table 2B, where

Synergistic Index=Qₐ/Q_{A}+Q_{b}/Q_{B} [Equation No. 1]

where
Qₐ=the quantity of component A used in a binary mixture that gives the desired effect
Q_{A}=the quantity of component A which when used alone gives the desired effect
Q_{b}=the quantity of component B used in a binary mixture that gives the desired effect
Q_{B}=the quantity of component B which when used alone gives the desired effect

Using these criteria, Synergistic Index was calculated for Trial of three compositions of BBIT and IPBC. The results of these calculations are presented in Table 2B below.

TABLE 2B

| | Compo- | | | | | | | Syner- | |
|---|---|---|---|---|---|---|---|---|---|
| | sition | | | | | Qₐ/ | Q_{b}/ | gistic | |
| Trial | Number | Qₐ | Q_{A} | Q_{b} | Q_{B} | Q_{A} | Q_{B} | Index | Comment |
| 10 | 18 | 0.15 | 0.23 | 0.09 | 0.48 | 0.65 | 0.19 | 0.84 | Synergy for |
| | | | | | | | | | Penicillium |
| | | | | | | | | | funiculosum |
| 11 | 21 | 0.37 | 0.7 | 0.22 | 1.2 | 0.53 | 0.18 | 0.71 | Synergy for |
| | | | | | | | | | Trichoderma |
| | | | | | | | | | virens |
| 12 | 24 | 1.38 | 5.4 | 0.81 | 1.5 | 0.25 | 0.54 | 0.795 | Synergy for |
| | | | | | | | | | Chaetosphaer- |
| | | | | | | | | | idium globosum |
| 13 | 27 | 0.13 | 0.29 | 0.07 | 0.47 | 0.65 | 0.19 | 0.84 | Synergy for |
| | | | | | | | | | Penicillium sp. |
| 14 | 30 | 0.37 | 0.42 | 0.23 | 2.4 | 0.88 | 0.09 | 0.97 | Synergy for |
| | | | | | | | | | Caratocystis |
| | | | | | | | | | Pilifera |

The Synergistic Indexes shown in Table 2B for compositions of the invention are evidence that mixtures of IPBC and BBIT exhibit a synergistic effect against a wide range of fungi.

While certain embodiments of the invention have been described above with particularity, it will be recognized that various modifications of the described embodiments will occur to those skilled in the art whose study this application. Such modifications are also within the scope of the appended claims.

That which is claimed is:

1. A microbicidal composition having a synergistic antifungal effect, which composition comprises:
(a) 3-iodo-2-propynyl-N-n-butylcarbamate; and
(b) 2-n-N-butyl-1,2-benzisothiazolin-3-one;
in which composition (a) and (b) are present in a proportion that exhibits a synergistic antifungal effect against Aspergillus niger, Penicillium funiculosum, Trichoderma virens, Chaetosphaeridium globosum, Penicillium sp., or Caratocystis Pilifera.

2. The composition of claim 1 in which (a) and (b) are present in a proportion that exhibits a synergistic antifungal effect against Aspergillus niger.

3. The composition of claim 1 in which (a) and (b) are present in a proportion that exhibits a synergistic antifungal effect against Penicillium funiculosum.

4. The composition of claim 1 in which (a) and (b) are present in a proportion that exhibits a synergistic antifungal effect against Trichoderma virens.

5. The composition of claim 1 in which (a) and (b) are present in a proportion that exhibits a synergistic antifungal effect against Chaetosphaeridium globosum.

6. The composition of claim 1 in which (a) and (b) are present in a proportion that exhibits a synergistic antifungal effect against Penicillium sp.

7. The composition of claim 1 in which (a) and (b) are present in a proportion that exhibits a synergistic antifungal effect against Caratocystis Pilifera.

8. The composition of claim 1 in which the proportion of (a):(b) is at least about 0.3 by weight.

9. The composition of claim 8 in which the proportion of (a):(b) is in the range of about 0.6 to about 7 by weight.

10. The composition of claim 9 in which the proportion of (a):(b) is in the range of about 1 to about 3 by weight.

11. The composition of claim 1 which includes a carrier.

12. A method for protecting a substrate from fungal infestation by, which method comprises:
treating a substrate with an antifungal amount of a composition including:
(a) 3-iodo-2-propynyl-N-n-butylcarbamate; and
(b) 2-n-N-butyl-1,2-benzisothiazolin-3-one;
in which composition (a) and (b) are present in a proportion that exhibits a synergistic antifungal effect against Aspergillus niger, Penicillium funiculosum, Trichoderma virens, Chaetosphaeridium globosum, Penicillium sp., or Caratocystis Pilifera.

13. A method for inhibiting fungal growth in a metal working fluid, which method comprises:
adding to the metal working fluid (a) 3-iodo-2-propynyl-N-n-butylcarbamate and (b) 2-n-N-butyl-1,2-benzisothiazolin-3-one, to produce a protected metal working fluid in which method (a) and (b) are present in a proportion that exhibits a synergistic antifungal effect against Aspergillus niger, Penicillium funiculosum, Trichoderma virens, Chaetosphaeridium globosum, Penicillium sp., or Caratocystis Pilifera.

14. A method for inhibiting fungal growth in on or a polymeric material, which comprises:
incorporating in a polymeric material (a) 3-iodo-2-propynyl-N-n-butylcarbamate, and (b) 2-n-N-butyl-1,2-benzisothiazolin-3-one;
in which method (a) and (b) are incorporated in the polymeric material in a proportion that exhibits a synergistic antifungal effect against Aspergillus niger, Penicillium funiculosum, Trichoderma virens, Chaetosphaeridium globosum, Penicillium sp., or Caratocystis Pilifera.

15. A method for inhibiting fungal growth in a dry film coating, which method comprises:
adding to a film-forming coating precursor (a) 3-iodo-2-propynyl-N-n-butylcarbamate, and (b) 2-n-N-butyl-1,2-benzisothiazolin-3-one; and
exposing the coating precursor to an oxygen-containing gas to form a dry film coating;
in which method (a) and (b) are present in the dry film coating in a proportion that exhibits a synergistic antifungal effect against Aspergillus niger, Penicillium funiculosum, Trichoderma virens, Chaetosphaeridium globosum, Penicillium sp., or Caratocystis Pilifera.

16. The method of claim 15 in which the proportion of (a):(b) is in the range of about 0.6 to about 7 by weight.

17. The method of claim 16 in which the proportion of (a):(b) is in the range of at least about 0.3 by weight.

18. The method of claim 17 in which the proportion of (a):(b) is in the range of about 0.6 to about 7 by weight.

19. The method of claim 18 in which the proportion of (a):(b) is in the range of about 1 to about 3 by weight.

20. The method of claim 16 which includes a carrier.
